SEQUENCE LISTING (25 sequences)

SEQ ID NO: 1 - DNA, 1053 bp, Lactobacillus plantarum
  misc_feature (1)..(1053): Lactobacillus plantarum 476LL 20 bi, LP01, deposit number LMG P-21021, phosphonate ABC transporter substrate-binding protein (PhnD)
atgtcaaaagttagtaagtcattattaggtctttcattcatttcggttttaggtttaggactcgcaggttgcgctagcggaaccagtgctaaatctggtggtagcagtacggatagcagcaaaaccatcacggtggtcttctatcctaacgaatcagccaagagttttgctggttcccgtacggccattaagcaagcagttgaaaaggcaacgggtaagacggttaaattacaaaccacgaccgactataacgttgcgatccaagcaattgcatccggcaaagctcaagtcgcatacatgggggctaacggttatatccaagcacaccacaagagcaaagatgttgtaccttttgccgcacaatctgatgctaaaggtaccttgaaagacgctacttataactcctacttaatggtacaacaaaaagatgccaaaaagtatgaagaaaacggtaagtacagtattaagaacgtcaaaggcaagaagatgtcttatgtttccaatagttcaacttcaggcttcttagtaccaacggctgaaatctcacgtgaattcaacatcaaaaccgctgaccaagataaattaactcaaagcggatcattctttagtaaagtgctctatggtggctctcatcagggttccgcggtcaacttactcaagggggatgcggatgtcgctgcctttgatgatgctgatttaatgccatacttaaacgttaccaagggatcatgggacaaggtcggctcaactttcactgttaaggataacgccgaagcgccatttacaagtttagccggcaagtcggtcgttaacattgccatgatgcctgttcaacaaggcccatgggtatacaataccaagtccctcagcaaggatgatcaaaagaagatcgccactgagttcacgtcgaagtcctttgcccaaaacaaaaagatcttctctgaaccaaatgcgaagacaccaatgatgttccctaagaaatcagaaaagtccaaattagttaatgttaccgacaagtggtacgcgccaacgcataagttagtaggttactaa

SEQ ID NO: 2 - DNA, 771 bp, Lactobacillus plantarum
  misc_feature (1)..(771): Lactobacillus plantarum 476LL 20 bi, LP01, deposit number LMG P-21021, phosphonate ABC transporter ATP binding protein (PhnC)
atgctcaaggttattcagctagataaaacatatggttccaataagcattcgctcaaagcagtcaacttcacagctaaaccgggcgaagtaactgccatcatcggcccgtctggtgctggtaaaacaactattttacggagtatcaatcagttgatccgcgatgatagtggtcaaattttgctcgatgacactgacattcgtcaagcgaacaaagctgaattacgcaaagttcgtcatcatatcggcatgattttccaaaactacaatctgatcagtccgttaacggcccttgaaaatgttctgcatggccgcttaggcgctaaatcaacggttgctggcatgctcggcctttacagtagcgccgaaaaacaagaggcgctacaactactcgacgaagtagggttaaaagaatacgcctaccaacgttgcgatcaattaagtggtggccaacaacaacgggtcgggatcgcacgggcattaatgcaacatcctaagatgattttatgtgatgaaccgattgcttccctagatcccaaatcaacgaccattgtcatggacattctacggcgattggcaaaggaaaaacaactgattatcctcattaatctacatcaggtcgatatcgcaatggcgtacaccgaccatatcgtcggcattaacagcggcgccatcgttttcgaaggcgccactaatgaagttgacgacgcggtcttacaacacatttatcgccaacctgaccaatcaacggcggtggctgccaatgaaaactaa

SEQ ID NO: 3 - DNA, 804 bp, Lactobacillus plantarum
  misc_feature (1)..(804): Lactobacillus plantarum 476LL 20 bi, LP01, deposit number LMG P-21021, phosphonate ABC transporte permease protein (PhnE2)
atgaaaactaatccaatgacaccgcaacgcttttttcagcaacgtcggttgcgattatcagttgttttgctcattttaattggcatctacgtcttatccatggccctcgttaattttcaggcgtgggcttcgatcagtaagattccagcgggcttaatgtggttgtttaccaactttattccgacaagtcgttcgatttcttatctgggaccgattctttatcagctgtggcggaccttgctcgtggcaatttcatcaaccatggtcgctagtctcttcgccctgatctttgcgattctcggtgctaagacgaccaccccaacacctgtcctccgctggattattcgttttggtgcttcactcttacgtaatattccggtggtcgcttgggcgatgattcttctgttttcttttaaacagagtgactttacgggcttcttggcactgttctttatgacgctaggctatctaacacgggcctttactgaaactattgaggacctcgatgccgaaaagcttcaagcactgcaagccgttggcgccaactatttccaatgtgtcttctgtggggtgttacccgaagccgctagtaccttgactagttggattctctacatgattgaaaacaacttacgggacgcaaccttggtcggactcttgacgggtacgggtgttggttttctctttgactactattttaaggcgttccgctatgacgctgctggtctaattgttttgttaattgcgattttagtcattgttttagaattgacatctaatcgaattcggagggcaattgcatga

SEQ ID NO: 4 - DNA, 855 bp, Lactobacillus plantarum
  misc_feature (1)..(855): Lactobacillus plantarum 476LL 20 bi, LP01, deposit number LMG P-21021, phosphonate ABC transporte permease protein (PhnE1)
atgattgaaaacagtcaacccaaaacacaagtacttcaaccgtcagcaccaccaacccggtcgatttcgttggtgactcggccccgcaaaatcctgcgagccgttatgattggcctaacgcttatcagtgtctattcattagtcacactcaataatgccaacctgaacatcattgacgctttgcatagcttaggtctcaacctgaatgctatgttcttacaccctagcgttggtcaagacacactggggcaactcttacgggccctaatgactagtgtttcattagcaatgttgacgactttgttaggagcgctaatcgccttcttcattgccgtcggtgctgcccgtaacttagcacccagttggttagctactagtattaaagcagtcatggcctttattcgtgccattccaacgattttatgggtgttaatttactccgtagtcatgggattaggtgctagtgccgccgtggtcggcttgaccttccacagtgtagcgtacttggttaaggcttattctgaaagtattgaagaaacctcacaagatacgattgaagcgttaaaggcgagtggcgtgggtttttggccgattgtttttcaggccattctcccgtcaatcattccagcacttcttagctggacgttcatccggtttgaaattaactttgccaatgcggtcgccgttggtgctgcagccggtgctgacgatatcggttactacctcttcatggctggcagtttctacttcgatttccatgaagttggtttgattgtctacctactcttgggtgtcgcaatcgtgctcgaactggtttctatgcggttacgtggccactacttaaagaacaactaa

SEQ ID NO: 5 - DNA, 798 bp, Lactobacillus plantarum
  misc_feature (1)..(798): Lactobacillus plantarum 476LL 20 bi, LP01, deposit number LMG P-21021, phosphonoacetaldehyde hydrolase
atgacaattaaagcagtaatttttgactgggccggcacgaccattgactatggcagccgggcgcccatcgtggctttccaaaaagcctttgccaacgttggtattcaaatctctgaagctgagattcgccaagatatgggactagataagtacacccatatccataaaatcatggatttaccggccgttcaaaacgactggcaggcgcgtttccaagtcttaccgactgaagacgactgcaatcaaatctttagcaacttcaaggcaatcttgcttagctccctgactgaattcggacaattaaagcccggcatgtcagctgtaattgactatttgaccgctcacaatatcagctatggcacgaccaccggctatgacgctgaaatgttagccctggtcttaccgatcgcagctaagcaaggctatcgtcccgccgtcaacatcacttcagaacaaactggcggtgttggtcgaccagcgccggcaatgcttgcgttagcagctgaacagctcacagtcaccgatccaacgaccgtcatgaaaattggggattccgttaatgatatcttagaaggcaacaacgccgacgcagtctctgtagggattattgatggtagcaacatcatgggactttcggagttagccttcaacgcccttagccccgccgaacaagctgaacgacgagcccacgtcactgccgcttatcaacgcgctggtgccgattacattttacaatcgatggctgaattaccggcattgcttgatcaaatcaatcaaccggttgccactgatcattag

SEQ ID NO: 6 - DNA, 1101 bp, Lactobacillus plantarum
  misc_feature (1)..(1101): Lactobacillus plantarum 476LL 20 bi, LP01, deposit number LMG P-21021, 2 aminoethylphosphonate: pyruvate aminotransferase
atgaaacaaccttatttactcctaacacccggtccactcagtacgactgccagtgttaaggacgcaatgcaaattgattattgtacttgggatagtgactatcggcacgtcaccgaaaccattcggcaacaaatcttagctatggcccaagccaatccagaaaactatacgaccgtcctactacaaggaagcggcagttttggtgttgaggccaccattggcacagcagtaccacggaccgatgcaactttaatgattgccattaacggcgcatatgggcatcggatcagtcaaattgcagaatattacgatattccacatatcgacgttgtgttcaacgaagatgaagccgtcgatccggttcggattgaagcaaccttagctgatcatccggagattacccacttcgcaaccgttcatagcgaaacgacaactgggattctcaacccaatcgaagccttaatgcctatcatgcatgaacacgagatcgtaacgatcatcgatgccatgtccagtttaggtggcgtcccgattagcattgatgaattggattgtgactacttgattagtagtgccaacaagtgtgtgcaaggcgtgcccggttttgcgttcattattgcaaaacaagcgaccttggcccacactgctgacaacgctcgttcactttgtctagatctctacgaccaatggcaagccatgaccaagcaaccaggaaaatggcgcttcacttcgccaacccacgtggtacatgcctttgcgcaagcactcattgaattgcaagcagaaggcggcgtgacaccacgctaccaacgttaccatgctagtcaacaactccttagcgacggcttacaagcgctaggttttgaattagtgattgatccagccattcaagggcccatcattacgtcattcaaatacccgaacgtcgactttgattttgctgacttctatcagttcatcaagcaacgcggcttcgtcatttatcctgggaaggtttcaaatatccctagcttcagaattggtaccatcggtgatgttgatacaactgatattcaacgactcttaacaattatcggtgattatcaacaacttcaccgctaa

SEQ ID NO: 7 - DNA, 1053 bp, Lactobacillus plantarum; identical to SEQ ID NO: 1
  misc_feature (1)..(1053): Lactobacillus plantarum 776 /1 bi, LP02, deposit number LMG P-21020, phosphonate ABC transporter substrate-binding protein (PhnD)

SEQ ID NO: 8 - DNA, 771 bp, Lactobacillus plantarum; identical to SEQ ID NO: 2
  misc_feature (1)..(771): Lactobacillus plantarum 776 /1 bi, LP02, deposit number LMG P-21020, phosphonate ABC transporter ATP binding protein (PhnC)

SEQ ID NO: 9 - DNA, 804 bp, Lactobacillus plantarum; identical to SEQ ID NO: 3
  misc_feature (1)..(804): Lactobacillus plantarum 776 /1 bi, LP02, deposit number LMG P-21020, phosphonate ABC transporte permease protein (PhnE2)

SEQ ID NO: 10 - DNA, 855 bp, Lactobacillus plantarum; identical to SEQ ID NO: 4
  misc_feature (1)..(855): Lactobacillus plantarum 776 /1 bi, LP02, deposit number LMG P-21020, phosphonate ABC transporte permease protein (PhnE1)

SEQ ID NO: 11 - DNA, 798 bp, Lactobacillus plantarum; identical to SEQ ID NO: 5
  misc_feature (1)..(798): Lactobacillus plantarum 776 /1 bi, LP02, deposit number LMG P-21020, phosphonoacetaldehyde hydrolase

SEQ ID NO: 12 - DNA, 1101 bp, Lactobacillus plantarum; identical to SEQ ID NO: 6
  misc_feature (1)..(1101): Lactobacillus plantarum 776 /1 bi, LP02, deposit number LMG P-21020, 2 aminoethylphosphonate: pyruvate aminotrasferse

SEQ ID NO: 13 - DNA, 1053 bp, Lactobacillus plantarum
  misc_feature (1)..(1053): Lactobacillus plantarum LP14, deposit number DSM 33401, phosphonate ABC transporter substrate-binding protein (PhnD)
atgtcaaaagttagtaagtcattattaggtctttcattcatttcggttttaggtttaggactcgcaggttgcgctagcggaaccagtgctaaatctggtggtagcagtacggatagcagcaaaaccatcacggtggtcttctatcctaacgaatcagccaagagttttgctggttcccgtacggccattaagcaagcagttgaaaaggcaacgggtaagacggttaaattacaaaccacgaccgactataacgttgcgatccaagcaattgcatccggcaaagctcaagtcgcatacatgggggctaacggttatatccaagcacaccacaagagcaaagatgttgtaccttttgccgcacaatctgatgctaaaggtaccttgaaagacgctacttataactcctacttaatggtacaacaaaaagatgccaaaaagtatgaagaaaacggtaagtacagtattaagaacgtcaaaggcaagaagatgtcttatgtttccaatagttcaacttcaggcttcttagtaccaacggctgaaatctcacgtgaattcaacatcaaaaccgctgaccaagataaattaactcaaagcggatcattctttagtaaagtgctctatggtggctctcatcagggttccgcggtcaacttactcaagggggatgcggatgtcgctacctttgatgatgctgatttaatgccatacttaaatgttaccaagggatcatgggacaaggtcggctcaactttcactgttaaggataacgccgaagcgccatttacaagtttagccggcaagtcggtcgttaacattgccatgatgcctgttcaacaaggcccatgggtatacaataccaagtccctcagcaaggatgatcaaaagaagatcgccactgagttcacgtcgaagtcctttgcccaaaacaaaaagatcttctctgaaccaaatgcgaagacaccaatgatgttccctaagaaatcagaaaagtccaaattagttaatgttaccgacaagtggtacgcgccaacgcataagttagtaggttactaa

SEQ ID NO: 14 - DNA, 771 bp, Lactobacillus plantarum
  misc_feature (1)..(771): Lactobacillus plantarum LP14, deposit number DSM 33401, phosphonate ABC transporter ATP binding protein (PhnC)
atgctcaaggttattcagctagataaaacatatggttccaataagcattcgctcaaagcagtcaacttcacagctaaaccgggcgaagtaactgccatcatcggcccgtctggtgctggtaaaacaactattttacggagtatcaatcagttgatccgcgatgatagtggtcaaattttgctcgatgacactgacattcgtcaagcgaacaaagctgaattacgcaaagttcgtcatcatatcggcatgattttccaaaactacaatctgatcagtccgttaacggcccttgaaaatgttctgcatggccgcttaggcgctaaatcaacggttgctggcatgctcggcctttacagtagcgccgaaaaacaagaggcgctacaactactcgacgaagtagggttaaaagaatacgcctaccaacgttgcgatcaattaagtggtggccaacaacaacgggtcgggatcgcacgggcattaatgcaacatcctaagatgattttatgtgatgaaccgattgcttccctagatcccaaatcaacgaccattgtcatggacattctacggcgattggcaaaggaaaaacaactgattatcctcattaatctacatcaggtcgatatcgcaatggcgtacaccgaccatatcgtcggcattaacagcggggccatcgttttcgaaggcgccactaatgaagttgacgacgcggtcttacagcacatttatcgccaacctgaccaatcaacggcggtggctgccaatgaaaactaa

SEQ ID NO: 15 - DNA, 804 bp, Lactobacillus plantarum
  misc_feature (1)..(804): Lactobacillus plantarum LP14, deposit number DSM 33401, phosphonate ABC transporte permease protein (PhnE2)
atgaaaactaatccaatgacaccgcaacgcttttttcggcaacgtcggttgcgattatcagttgttttgctcattttaattggcatctacgtcttatccatggccctcgttaattttcaggcgtgggcttcgatcagtaagattccagcgggcttaatgtggttgtttaccaactttattccgacaagtcgttcgatttcttatctgggaccgattctttatcagctgtggcggaccttgctcgtggcaatttcatcaaccatggtcgctagtctcttcgccctgatctttgcgattctcggtgctaagacaaccaccccaacgcctgtcctccgctggattattcgttttggtgcttcactcttacgtaatattccggtggtcgcttgggcgatgattcttctgttttcttttaaacagagtgactttacgggcttcttggcactgttctttatgacgctaggctatctgacacgggcctttactgaaactattgaggacctcgatgccgaaaagcttcaagcactgcaagccgttggcgccaactatttccaatgtgtcttctgtggggtgttacccgaagccgctagtaccttgactagttggattctctacatgattgaaaacaacttacgggacgcaaccttggtcggactcttgacgggtacgggtgttggttttctctttgactactattttaaggcgttccgctatgacgctgctggtctaattgttttgttaattgcgattttagtcattgttttagaattgacatctaatcgaattcggagggcaattgcatga

SEQ ID NO: 16 - DNA, 855 bp, Lactobacillus plantarum
  misc_feature (1)..(855): Lactobacillus plantarum LP14, deposit number DSM 33401, phosphonate ABC transporte permease protein (PhnE1)
atgattgaaaacagtcaacccaaaacacaagtacttcaaccgtcagcaccaccaacccggtcgatttcgttggtgactcggccccgcaaaatcctgcgagccgttatgattggcctaacgcttatcagtgtctattcattagtcacactcaataatgccaacctgaacatcattgacgctttgcatagcttaggtctcaacctgaatgctatgttcttacaccctagcgttggtcaagacacactggggcaactcttacgggccctaatgactagtgtttcattagcgatgttgacgactttgttaggagcgctaatcgccttcttcattgccgtcggtgctgcccgtaacttagcacccagttggttagctactagtattaaagcagtcatggcctttattcgtgccattccaacgattttatgggtattaatttactccgtagtcatgggattaggtgctagtgccgccgtggtcggcttgaccttccacagtgtagcgtacttggttaaggcttattctgaaagtattgaagaaacctcgcaagatacgattgaagcgttaaaggcgagtggcgtgggtttttggccaattgtttttcaggccattctcccgtcaatcattccagcacttcttagctggacgtttatccggtttgaaattaactttgccaatgcggtcgccgttggtgctgcagctggtgctgacgatatcggttactacctcttcatggctggcagtttctacttcgatttccatgaagttggtttgattgtctacctactcttgggtgtcgcaatcgtgctcgaactggtttctatgcggttacgtggccactacttaaagaacaactaa

SEQ ID NO: 17 - DNA, 798 bp, Lactobacillus plantarum
  misc_feature (1)..(798): Lactobacillus plantarum LP14, deposit number DSM 33401, phosphonoacetaldehyde hydrolase
atgacaattaaagcagtaatttttgactgggccggcacgaccattgactatggcagccgggcgcccatcgtggctttccaaaaagcctttgccaacgttggtattcaaatctctgaagctgagattcgccaagatatgggactagataagtacacccatatccataaaatcatggatttaccggccgttcaaaacgactggcaggcgcgtttccaagtcttaccgactgaagacgactgcaatcaaatctttagcaacttcaaggcaatcttgcttagctccctgactgaattcggacaattaaagcccggcatgtcagctgtaattgactatttgaccgctcacaatatcagctatggcacgaccaccggctatgacgctgaaatgttagctctggtcttaccgattgcagctaagcaaggctatcgtcccgccgtcaacatcacttcagaacaaactggtggtgttggtcgaccagcgccggcaatgcttgcgttagcagctgaacagctcacaatcaccgatccaacgaccgtcatgaaaattggggattccgttaatgatatcttagaaggcaacaacgccgacgcagtctctgtagggattattgatggtagcaacatcatgggactttctgagttagccttcaacgcccttagccccgccgaacaagctgaacgacgagcccacgtcactgccgcttatcaacgcgctggtgccgattacattttacaatcgatggctgaattaccggcattgcttgatcaaatcaatcaaccggttgccactgatcattag

SEQ ID NO: 18 - DNA, 1101 bp, Lactobacillus plantarum
  misc_feature (1)..(1101): Lactobacillus plantarum LP14, deposit number DSM 33401, 2 aminoethylphosphonate: pyruvate aminotrassferase
atgaaacaaccttatttactcctaacacccggtccactcagtacgactgccagtgttaaggacgcaatgcaaattgattattgtacttgggatagtgactatcggcacgtcaccgaaaccattcggcaacaaatcttagctatggcccaagccaatccagaaaactatacgaccgtcctactacaaggaagcggcagttttggtgttgaggccaccattggcacagcagtaccacggaccgatgcaactttaatgattgccattaacggcgcatatgggcatcggatcagtcaaattgcagaatattacgatattccacatatcgacgttgtgttcaacgaagatgaagccgtcgatccggttcggattgaagcaaccttagctgatcatccggagattacccacttcgcaaccgttcatagcgaaacgacaactgggattctcaacccaatcgaagccttaatgcctatcatgcatgaacacgggatcgtaacgatcatcgatgccatgtccagtttaggtggcgtcccgattagcattgatgaattggattgtgactacttgattagtagtgccaacaagtgtgtgcaaggcgtgcccggttttgcgttcatcattgcaaaacaagcgaccttggcccacactgctgacaacgctcgttcactttgtctagatctctacgaccaatggcaagccatgaccaagcaaccaggaaaatggcgcttcacttcgccaacccacgtggtacatgcctttgcgcaagcactcattgaattgcaagcagaaggcggcgtgacaccacgctaccaacgttaccatgctagtcaacaactccttagcgacggcttacaagcgctaggttttgaattagtgattgatccagccattcaagggtccatcattacgtcattcaaatacccgaacgtcgactttgattttgctgacttctatcagttcatcaagcaacgcggcttcgtcatttatcctgggaaggtttcaaatatccctagcttcagaattggtaccatcggtgatgttgatacaactgatattcaacgactcttaacaattatcggtgattatcaacaacttcaccgctaa

SEQ ID NO: 19 - DNA, 1053 bp, Lactobacillus pentosus
  misc_feature (1)..(1053): Lactobacillus pentosus LPS01, deposit number DSM 21980, phosphonate ABC transporter substrate-binding protein (PhnD)
atgtcaaaagtaagtaagtctttattaggtctttcgttcatttcagttttaggtttgggtcttgcaggttgtgccagcggaaccagcgcacaatccggtagcgacagttcggataagagcaaaacgattacggtcgtcttctatccaaacgaatcggccaagagttttgccggctcacggacagcaatcaagaaagctgttgaaaaagccactggcaagacggtcaagctacaaacgaccacggattacaacgttgcgattcaagcgattgcttcaggtaaagcccaagtcgcttacatgggggccaacggttatatccaagcacatcacaagagcaaggacgttgtgccattcgccgcacaatctgatgctaaggggacgttgaaagacgcaacctataactcatacttaatggtacaacaaaaagacgccaaaaagtatgaagaaaacggtaagtacagtattaagaatgttaagggcaagaaaatgtcctacgtttcaaacagttccacttctggtttcttagtgccaacggctgaaatctcccgtgaatttaacattaagacatccgaccaagataagttgacgcaaagcggccaattcttcagtaaagtactttacggtggttcacaccaaggctcagccgttaacttgatcaagggtgacgcagacgttgccgccttcgatgacgctgacttgatgccatatttgaaagtcactaagggctcatgggacaaagttggttcaacgttcacggtcaaagacaatgccgacgcaccattcacgagcttaactggcaagtccgttgtcaacatcgcgatgatgccagttcaacaaggcccatgggtatacaacaccaaggcactcagcaaggacgaccaaaagaagatcgctactgaatttacatcaaaatcatttgcacaaaacaagcagatcttctcagaaccaaacgccaagacaccaatgatgttccctaagaagtctgacaagtctaaattagttaaagttaccgacaaatggtacgcgccaacacacaagttagtgggttactaa

SEQ ID NO: 20 - DNA, 771 bp, Lactobacillus pentosus
  misc_feature (1)..(771): Lactobacillus pentosus LPS01, deposit number DSM 21980, phosphonate ABC transporter ATP binding protein (PhnC)
atgctcaaggttattcagctagataaaacttacggttccaataagcactcgctcaaagccgtcaacttcacggctaaacccggtgaagtgactgccatcattgggccatccggtgctggtaagacgacgattttacggagtatcaatcaattaattcgcgatgacagtggtcagattttgctcgatgatacggacattcgccaggccaacaagtccgagttgcggaagattcgtcaccacattggcatgattttccaaaattacaatttgatcagtccattgacggccttagaaaacgtcttacatggccgtttgggcgctaagtcaacggttgctggcatgctcggcttgtacagtcctgccgaaaagcaggaagccctacaactactcgatgaagttggtttgaaagaatatgcttatcaacgttgcgaccaattaagtggtggtcaacaacaacgggtcgggattgcgcgtgcactcatgcagcatccgaaaatgattctgtgtgacgaaccgattgcgtcacttgatccgaagtccacgactatcgtaatggatattttgcggcgtttagcgaaggaaaaacaactcattattctcattaatttacatcaagttgatattgcgatggcttacacggatcacattgttggcatcaacagcggcgccatcgtctttgaaggcgctacgaaggatgttgacgatgccgtcttacaagcgatttatcgccagtcggagcaaccagcgacggtgactgccaatgaaaactaa

SEQ ID NO: 21 - DNA, 804 bp, Lactobacillus pentosus
  misc_feature (1)..(804): Lactobacillus pentosus LPS01, deposit number DSM 21980, phosphonate ABC transporte permease protein (PhnE2)
atgaaaactaatccaatgactccggaacggtttttccggcaacggcgtctaagcctatccgtcgtcctactcatcttgattgggatttacgttatctcgatggcattggtcaattttcaggcctgggcatcgatcagtaaaattccagccgggttaatgtggttgtttacgaactttattccgacgagtcgctcgatttcttatctaggaccgattctctatcagctgtggcggaccttgcttgtggcaatttcatcaacaatggtcgccagtctattcgccctggtatttgccattctgggtgcgaaaacgacgaccccgacgccagcgctccgctggggcattcgcttcggtgcttccctattgcggaatatcccagtggtcgcttgggcgatgattctcttgttttcgttcaagcagagtgatttcaccggctttttagccctcttcttcatgacattgggttacttgacccgggcctttacggaaacgattgaagacctggacgctgaaaagctgcaagcactccaagccgttggcgccaattattttcaatgcgttttttgcggcgtgttgcccgaagctgctagtaccttgaccagctgggtgctctacatgattgaaaacaatttgcgagatgcgactttagttggcttactgactggaacgggagtcgggttcctgtttgactactatttcaaggcctttcgttacgatgcggccggcctgattgttttgttaattgcgatcttagtcattgtcttagaattaacttctaatcgaattcggagggcgattgcatga

SEQ ID NO: 22 - DNA, 855 bp, Lactobacillus pentosus
  misc_feature (1)..(855): Lactobacillus pentosus LPS01, deposit number DSM 21980, phosphonate ABC transporte permease protein (PhnE1)
atgattgaaaatagtcaaccaaaaccacaagtgctccacccgtcagcgccacccactaatcgaatttcgctagtgacgaagccccgtaaaattctgcgcggcgtcatgctgagtttggcactgatcaccatctattcactagtcacgctcaacacggcgaacttagcgattagcgatgcgttacacagtttaggccttaatctgaacgccatgttcttgcatccaagcattggtcaagatacttggccacagctcttacgggccctactaaccagtgtttcactcgcaatgttaaccaccttactaggtgcactgattgcctttttcatcgcactcggagctgcacgtaacttagccccgagctggcttgcaaccagtatcaaagcagtcatggccttcatccgcgcgattcccacaattttgtgggtcctgatttattcagtcgtgatgggtcttggcgccagcgctgcggtggttgggctcacgttccacagtgtcgcttacctggtcaaggcttactctgaaagtattgaggaaacttcagcagataccatcgaagccctcaaggccagcggggttggcttctggccaatcgtttttcaggcaatcttgccttcaattattccggcactgctcagctggacgtttatccggtttgaaatcaactttgcaaatgccgtcgcagttggtgctgcggctggggctgatgatatcggctacttcctcttcatggcgggcagtttctacttcgacttccatgaggttggtttgattgtctacctactcttaggtgtcgcaatcgtactcgaattggtttctatgcggttacgcggacactatctgaagcaaaattaa

SEQ ID NO: 23 - DNA, 798 bp, Lactobacillus pentosus
  misc_feature (1)..(798): Lactobacillus pentosus LPS01, deposit number DSM 21980, phosphonoacetaldehyde hydrolase
atgacaattaacgcagtaatttttgattgggctggtaccacgattgattatggtagccgcgcaccaatcgttgcctttcaaaaggcctttgaaaatgttggtattcaaatctctgaagccgagattcgtcaagatatgggcctcgacaagtacacgcatatccataaaatcatggacttacctgccgttcaaaacgactggcaagcacgctttcaagtcttacctactgaagatgactgcaatcaaattttcagtgacttcaaggccatcttactgagctcattgacagaatttggtcagttgaaacctggtatgtctgaggtgattgactatttgaacacgcaacatatcagctatgggacgacgactggctacgatgccgaaatgctggcactggtcttgccaatcgccgcccgtcaaggctaccaaccagcggtcaacattacttctgaacaaaccaacggggtcggtcggcccgccccagatatgttggccttggctgctgaacaactcaacattactgacccaacgacggtcatgaaagttggcgattcggtcaatgacattctggaaggtaacaacgccaatgcggtgagtgtcggtatcattgatggcagcaatattatggggctctctgaaattgcctttaacgccctcagttctgccgaacaagctgaacgccgggcccaagtggccgctgaataccaacgtgcgggtgcggactacattctgcaatcgatggcggaactaccagcgttaatcgcgcaaatcaatcaacccgttgccactgatcattag

SEQ ID NO: 24 - DNA, 1101 bp, Lactobacillus pentosus
  misc_feature (1)..(1101): Lactobacillus pentosus LPS01, deposit number DSM 21980, 2 aminoethylphosphonate: pyruvate aminotrasferse
atgaaacaaccttatttactcttaacacctggcccactcagtactacggcgagtgttaaggatgccatgcaaatcgactactgcacatgggatagtgattatcgccaggtcactgaaacgattcgccagcagattctggccatggctcaagccgatgctgagacgtacacgacggtgctcctacaagggagtggtagttttggcgtcgaagcaacgattggaactgccgtaccacgcaccaaagccactttgatgattgccatcaacggggcgtatgggcaacgaatcagccaaattgctgaatattacggtattccgcacgttgacgtcgtcttcaatgaagatgaagccgttgacccagcacgcatcgaagcgaccttgactgcgcatccagaaatcacgcattttgccacggtccacagcgaaacgacgactgggattctaaatccgatcgaagcattgatgccaattatgcatgcacacggcatcgtaacgattatcgatgcgatgtctagcttaggcggcgttcccatcaatgtggatgaactcgactgtgattacctcatcagtagcgcgaacaagtgtgttcaaggggttccgggctttgccttcattattgccaagcaagcgaccttggcacaaaccgtcgacaatgcacgttcactctgcttagacttgtacgaccaatggcaagcgatgactaagcaaccgggtaagtggcgcttcacttccccaacccacgtggtccacgcctttgcacaagcgttaattgaattgcaagctgaaggcggcgtcgcaccacgttacgaacgctatcatgctaaccaacaattattgagtgacggtatgcaagcactcggatttgatttggtgattgatccagcaattcaaggcccaatcattacttcgtttaaatatccaaacactgatttcgattttgccgatttttatcagttcattaagcaacggggctttgtcatctaccctggtaaagtctccaacatccctagcttccgtattggaacaattggtgatgtagaaccttcagatatccaacggttactgacaattattggtgattaccaacaactgcatcaatag

SEQ ID NO: 25 - DNA, 294 bp, Lactococcus plantarum
  misc_feature (1)..(294): Lactococcus plantarum LPS01 deposit number DSM 21980, phosphonate ABC transporter ATP-binding protein PhnC
ttgacggccttagaaaacgtcttacatggccgtttgggggctaagtcaacggtagctggcatgctcggcgttgagagtcctgccgaagagcaggaagccctacccctactcgatgaagttggtttgaaagaatatgcttatcaacgttgcgaccaattaagtggtggtcaacaacagcgggtcgggattgcgcgtgcactcatgcagcatccgaaaatgattctgtgtgacgaaccgattgcgtcacttgatccgaagaccacgactatcgtaatagatattttgcggcgttta

(19) United States
(12) Patent Application Publication
(10) Pub. No.: US 2023/0293604 A1
(43) Pub. Date: Sep. 21, 2023

(54) USE OF BACTERIAL STRAINS LACTOBACILLUS BREVIS, LACTOBACILLUS PLANTARUM AND/OR LACTOBACILLUS PENTOSUS IN THE REDUCTION OF AN INGESTED OR ACCUMULATED AMOUNT OF PHOSPHONATES IN THE HUMAN MICROBIOTA

(71) Applicant: PROBIOTICAL S.P.A., Novara (NO) (IT)

(72) Inventors: Vera MOGNA, Novara (IT); Marco PANE, Novara (IT); Angela AMORUSO, Novara (IT)

(21) Appl. No.: 17/917,458

(22) PCT Filed: Apr. 9, 2021
(86) PCT No.: PCT/IB2021/052963

§ 371 Date: Oct. 6, 2022

(30) Foreign Application Priority Data
Apr. 9, 2020 (IT) 102020000007690

(51) Int. Cl. A61K 35/747; A61P 1/00

(52) U.S. Cl. CPC A61K 35/747; A61P 1/00

(57) ABSTRACT

The present invention relates to a mixture and a composition comprising said mixture, said mixture comprising or, alternatively, consisting of at least one isolated bacterial strain belonging to the species Lactobacillus brevis, optionally combined with at least one isolated bacterial strain belonging to the species Lactobacillus plantarum and/or Lactobacillus pentosus for use in a method for the preventive or curative treatment of a disorder or an ailment or a disease associated with an ingestion or an accumulation -through food - of phosphonates in the human or animal microbiota. Furthermore, the present invention relates to a use - preferably non-therapeutic - of a composition comprising said mixture, and at least one physiologically and/or pharmaceutically acceptable excipient or additive, in the reduction of an amount - ingested or accumulated through food - of phosphonates in the human or animal microbiota.

The present invention relates to a use - preferably non-therapeutic - of a mixture comprising or, alternatively, consisting of at least one isolated bacterial strain belonging to the species Lactobacillus brevis, and optionally, at least one isolated bacterial strain belonging to the species Lactobacillus plantarum and/or at least one isolated bacterial strain belonging to the species Lactobacillus pentosus in the reduction of an ingested or accumulated amount of phosphonates in the human microbiota.

Furthermore, the present invention relates to a use - preferably non-therapeutic - of a composition comprising said mixture, and at least one physiologically and/or pharmaceutically acceptable excipient or additive, in the reduction of an ingested or accumulated amount of phosphonates in the human microbiota.

Furthermore, the present invention relates to a mixture or a composition comprising said mixture, said mixture comprising or, alternatively, consisting of at least one isolated bacterial strain belonging to the species Lactobacillus brevis, and optionally at least one isolated bacterial strain belonging to the species Lactobacillus plantarum and/or at least one isolated bacterial strain belonging to the species Lactobacillus pentosus for use in a method for the preventive or curative treatment of a disorder or an ailment or a disease associated with an ingestion or an accumulation of phosphonates in the human microbiota.

Furthermore, the present invention relates to a mixture comprising or, alternatively, consisting of at least one isolated bacterial strain belonging to the species
(i) Lactobacillus brevis, wherein said strain is selected from the group comprising or, alternatively, consisting of Lactobacillus brevis LBR01, identified under deposit number DSM 23034, Lactobacillus brevis LBR02, identified under deposit number DSM 29404; and mixtures thereof;
optionally combined with at least one isolated bacterial strain belonging to the species Lactobacillus plantarum and/or at least one isolated bacterial strain belonging to the species Lactobacillus pentosus for use in a method for the preventive or curative treatment of a disorder or an ailment or a disease associated with an ingestion or an accumulation of phosphonates in the human microbiota.

Furthermore, the present invention relates to a non-therapeutic use - in the reduction of an ingested or accumulated amount of phosphonates in the human microbiota - of a mixture comprising or, alternatively, consisting of at least one isolated bacterial strain belonging to the species
(i) Lactobacillus brevis, wherein said strain is selected from the group comprising or, alternatively, consisting of Lactobacillus brevis LBR01, identified under deposit number DSM 23034, Lactobacillus brevis LBR02, identified under deposit number DSM 29404; and mixtures thereof;
optionally combined with at least one isolated bacterial strain belonging to the species Lactobacillus plantarum and/or at least one isolated bacterial strain belonging to the species Lactobacillus pentosus.

Furthermore, the present invention relates to an in vitro use - in the reduction of phosphonates, preferably of glyphosate - of a mixture comprising or, alternatively, consisting of at least one isolated bacterial strain belonging to the species

(i) Lactobacillus brevis, wherein said strain is selected from the group comprising or, alternatively, consisting of Lactobacillus brevis LBR01, identified under deposit number DSM 23034, Lactobacillus brevis LBR02, identified under deposit number DSM 29404; and mixtures thereof.

Preferably, the in vitro use allows to obtain a reduction of phosphonates comprised from 2% to 20%, preferably comprised from 2% to 14% with respect to the initial amount of phosphonates present.

Furthermore, the present invention relates to a mixture comprising or, alternatively, consisting of at least one isolated bacterial strain belonging to the species

(i) Lactobacillus brevis, wherein said strain is selected from the group comprising or, alternatively, consisting of Lactobacillus brevis LBR01, identified under deposit number DSM 23034, Lactobacillus brevis LBR02, identified under deposit number DSM 29404; and mixtures thereof, for use in the maintenance/restoration of the human microbiota homeostasis.

Furthermore, the present invention relates to a method for the reduction of an ingested or accumulated amount of phosphonates in the human microbiota, wherein said method provides for the use of a composition comprising at least one isolated bacterial strain belonging to the species Lactobacillus brevis, and optionally at least one isolated bacterial strain belonging to the species Lactobacillus plantarum and/or at least one isolated bacterial strain belonging to the species Lactobacillus pentosus, and at least one physiologically and/or pharmaceutically acceptable excipient or additive.

Phosphonates (or phosphonic acids) are organic phosphorus compounds, containing at least one C—PO(OR)₂ group, wherein each R substituent is independently selected from the group consisting of hydrogen, an alkyl group or an optionally substituted aryl group. Phosphonates are characterised by a covalent carbon-phosphorus bond (C-P bond) which is particularly stable, extremely resistant to chemical hydrolysis, to thermal decomposition and to photolysis. Phosphonates are compounds common in many natural substances, but they are also artificial products of a variety of human activities. The amounts of phosphonates introduced into the environment as a result of human activities (mainly as herbicides, or as plant growth regulators) have made phosphonates a concerning cause of pollution and toxicity precisely due to the high stability of the C-P bond.

The scientific publication Ju Kou-San et al., “Genomics-enabled discovery of phosphonate natural products and their biosynthetic pathways”, J Ind Microbiol Biotechnol 2013, Vol. 41, published online on 24 Nov. 2013 (24-11-2013), pages 345-356, DOI 10.1007/s10295-013-1375-2 relates to a study conducted to identify natural phosphonate products.

The scientific article Manav M. Cemre et al., “The Abc of Phosphonate Breakdown: A Mechanism for Bacterial Survival”, BioEssays 2018, Vol. 800091, pages 1-11, DOl: 10.1002/bies.201800091 relates to a study conducted to understand possible C-P bond breakdown mechanisms in phosphonate compounds.

The scientific publication Chin Jason P. et al., “Microbial transformations in phosphonate biosynthesis and catabolism, and their importance in nutrient cycling”, Current Opinion in Chemical Biology 2016, Vol. 31, pages 50-57, DOI: hiip://dx.doi.org/10.1016/j.cbpa.2016.01.010 relates to a study conducted to understand the reduced forms of phosphorous such as phosphonates.

For example, glyphosate (CAS No. 1071-83-6; N-(phosphonomethyl)glycine) is a phosphonate used as active ingredient in non-selective glyphosate-based herbicides (GBHs). It is estimated that, to date, generally about 9.4 million tons of GBHs have been distributed on crops all over the world. GBHs have always been considered not to pose a risk to human or animal health, given that glyphosate inhibits a key enzyme (5-enolpyruvylshikimate-3-phosphate) (or EPSPS) in plants, but there is no metabolic pathway promoted by such enzyme in mammalian cells. Nevertheless, currently there is an ongoing debate on the potential carcinogenic and non-carcinogenic, but nevertheless toxic, effects of glyphosate, given that GBHs could still have negative effects on mammalian biology.

In this regard, a very recent pilot study investigated the non-carcinogenic effects of glyphosate-based herbicides. This study found that exposure of female adult mice to amounts of GBHs considered to be in acceptable proportions for humans, causes a modification of the microbiota of pup mice before puberty, with currently unpredictable health consequences.

As a matter of fact, the gut microbiota plays a crucial role in maintaining the health of any individual, given that an inadequate (or polluted) or damaged or intoxicated bacterial flora may be the basis of several diseases, especially immune-modulated diseases.

Therefore, there arises the need to remedy the toxic and harmful effects caused by or originating from GBHs in food, in animals and in human beings. Therefore, it is essential to be able to find a validly applicable and useful solution that allows to safeguard the gut microflora from the potentially harmful and deleterious effects caused by or originating from ingestion of GBHs by animals and human beings through food. Last but not least, there arises the need for a validly applicable and useful solution capable of reducing an amount - ingested or accumulated through food - of phosphonates in the organism of an animal or human being and, therefore, to combat the potential negative effects of phosphonates, in the animal or human microbiota.

More precisely, following an extensive and intense research and development activity, the inventors of the present invention were able to identify and select, from a very large group of species of bacteria and bacterial strains, bacteria belonging to the species Lactobacillus brevis, Lactobacillus plantarum and/or belonging to the species Lactobacillus pentosus. Such bacteria having shown a marked ability to degrade phosphonates in human microbiota (plausibly through a cleavage of the C-P bonds thereof), and being well-tolerated and easy to administer to any category of subject, including paediatric subjects.

Thus, forming an object of the present invention is a use - preferably non-therapeutic - of a mixture comprising or, alternatively, consisting of at least one isolated bacterial strain belonging to the species Lactobacillus brevis, optionally combined with at least one isolated bacterial strain belonging to the species Lactobacillus plantarum and/or at least one isolated bacterial strain belonging to the species Lactobacillus pentosus in the reduction of an amount - ingested or accumulated through food - of phosphonates (for example, glyphosate (CAS No. 1071-83-6; N-(phosphonomethyl)glycine) in the human microbiota, having the characteristics as defined in the attached claims.

Furthermore, forming an object of the present invention is a use - preferably non-therapeutic - of a composition comprising said mixture, and at least one physiologically and/or pharmaceutically acceptable excipient or additive, in the reduction of an amount - ingested or accumulated through food - of phosphonates (for example, glyphosate (CAS No. 1071-83-6; N-(phosphonomethyl)glycine) in human microbiota, having the characteristics as defined in the attached claims.

Furthermore, forming another object of the present invention is a mixture and a composition comprising said mixture; said mixture comprising or, alternatively, consisting of at least one isolated bacterial strain belonging to the species Lactobacillus brevis, optionally combined with at least one isolated bacterial strain belonging to the species Lactobacillus plantarum and/or at least one isolated bacterial strain belonging to the species Lactobacillus pentosus for use in a method for the preventive or curative treatment of a disorder or an ailment or a disease associated with an ingestion or an accumulation - through food - of phosphonates (for example, glyphosate (CAS No. 1071-83-6; N-(phosphonomethyl)glycine) in the human or animal microbiota, having the characteristics as defined in the attached claims.

Forming an object of the present invention is a use - preferably non-therapeutic - of a mixture comprising or, alternatively, consisting of at least one isolated bacterial strain belonging to the species Lactobacillus brevis, preferably said at least one bacterial strain being the bacterial strain Lactobacillus brevis LBR01 identified under deposit number DSM 23034, in the reduction of the amount - ingested or accumulated through food - of phosphonates (for example, glyphosate (CAS No. 1071-83-6; N-(phosphonomethyl)glycine) in the human microbiota.

Furthermore, forming an object of the present invention is a use - preferably non-therapeutic - of a composition comprising said mixture, comprising or alternatively consisting of at least one isolated bacterial strain belonging to the species Lactobacillus brevis, preferably said at least one bacterial strain being the bacterial strain Lactobacillus brevis LBR01 identified under deposit number DSM 23034, and at least one physiologically and/or pharmaceutically acceptable excipient or additive, in the reduction of an amount - ingested or accumulated through food - of phosphonates (for example, glyphosate (CAS No. 1071-83-6; N-(phosphonomethyl)glycine) in the human microbiota.

Furthermore, forming an object of the present invention is a mixture and a composition comprising said mixture; said mixture comprising or, alternatively, consisting of at least one isolated bacterial strain belonging to the species Lactobacillus brevis, preferably said at least one bacterial strain being the bacterial strain Lactobacillus brevis LBR01, identified under deposit number DSM 23034 for use in a method for the preventive or curative treatment of a disorder or an ailment or a disease associated with an ingestion or an accumulation - through food - of phosphonates (for example, glyphosate (CAS No. 1071-83-6; N-(phosphonomethyl)glycine) in the human or animal microbiota, having the characteristics as defined in the attached claims.

Furthermore, the present invention relates to a method for the reduction of an ingested or accumulated amount of phosphonates in the human microbiota, where said method provides for the administration of a composition comprising at least one isolated bacterial strain belonging to the species Lactobacillus brevis, preferably said at least one bacterial strain being the bacterial strain Lactobacillus brevis LBR01, identified under deposit number DSM 23034, and at least one physiologically and/or pharmaceutically acceptable excipient or additive.

Furthermore, forming an object of the present invention is a method for the preventive or curative treatment of a disorder or an ailment or a disease associated with an ingestion or an accumulation - through food - of phosphonates (for example, glyphosate (CAS No. 1071-83-6; N-(phosphonomethyl)glycine) in the human or animal microbiota, said method comprising the administration of a composition comprising at least one isolated bacterial strain belonging to the species Lactobacillus brevis, preferably said at least one bacterial strain being the bacterial strain Lactobacillus brevis LBR01, identified under deposit number DSM 23034, and at least one physiologically and/or pharmaceutically acceptable excipient or additive.

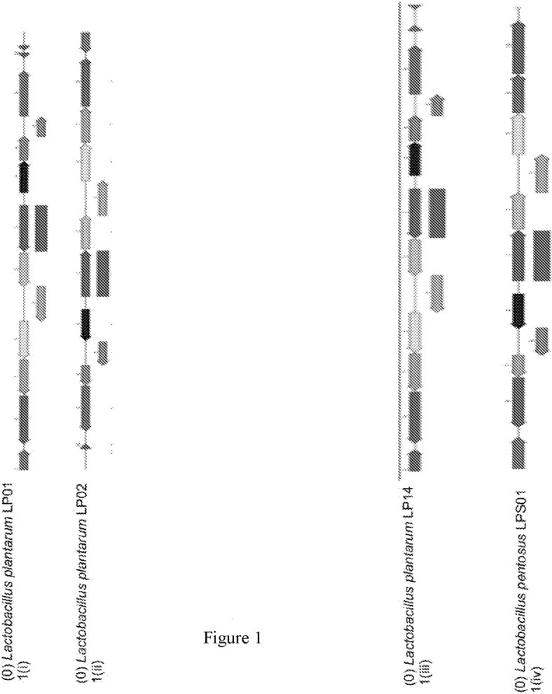

The present invention will now be described hereinafter with the aid of the attached drawings, wherein FIGS. 1.(i) to 1.(iv) show schematisations of the gene sequences - respectively of the bacterial strains:
1.(i) Lactobacillus plantarum 476LL 20 bi (LP01; identified under deposit number LMG P-21021),
1.(ii) Lactobacillus plantarum 776/1 bi (LP02; identified under deposit number LMG P-21020),
1.(iii) Lactobacillus plantarum (LP14; identified under deposit number DSM 33401), and
1.(iv) Lactobacillus pentosus (LPS01; identified under deposit number DSM 21980),
all of the above, responsible for encoding certain proteins involved in the degradation of phosphonates (for example, glyphosate (CAS No. 1071-83-6; N-(phosphonomethyl)glycine).

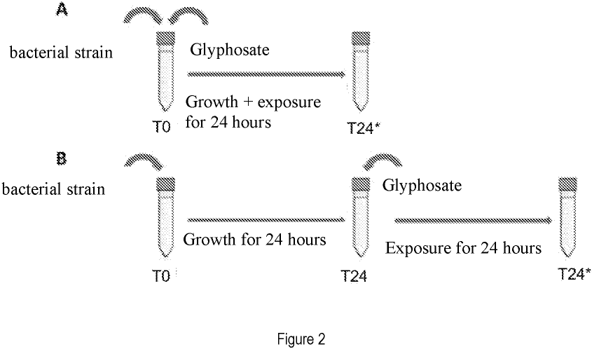

FIG. 2 shows the scheme of the two methods used for the evaluation of the reduction action of phosphonates of the bacterial strains according to the present invention. In particular, in method A the reduction action of the phosphonates was evaluated during microbial growth, while in method B, it was evaluated following the accumulation of biomass (B). T24* is the time after 24 hours of exposure to glyphosate.

Therefore, in said mixture (and said composition) there could be present at least one isolated bacterial strain belonging to the species Lactobacillus plantarum, or there could be present at least one isolated bacterial strain belonging to the species Lactobacillus brevis, or there could be present at least one isolated bacterial strain belonging to the species Lactobacillus pentosus, or there could be present a combination of at least one isolated bacterial strain belonging to the species Lactobacillus brevis, of at least one isolated bacterial strain belonging to the species Lactobacillus plantarum and of at least one isolated bacterial strain belonging to the species Lactobacillus pentosus. Once administered and ingested by an animal or a human being (for example in paediatric age), such mixture (or said composition) is thus capable of reducing the amount of phosphonates previously ingested or accumulated - through food - in the animal or human microbiota.

Surprisingly, the isolated bacterial strains described herein, in particular the bacterial strain Lactobacillus brevis LBR01, identified under deposit number DSM 23034, have shown a marked ability to degrade phosphonates (for example glyphosate). This ability is particularly appropriate given the possible harmful effects linked to these substances.

Furthermore, this ability results in an increase in the nutritional quality of the ingested foods. This because it allows to improve the absorption by the gut microbiota.

As a matter of fact, it has been surprisingly found that the bacterial strains described in the present context, for example the isolated bacterial strain Lactobacillus brevis LBR01, identified under deposit number DSM 23034, may be used to reduce the amount of phosphonates present in foods. This leads to an increase in the nutritional properties of foods taken and improves the absorption thereof.

Furthermore, it was observed that the specific strains described herein may be used to degrade phosphonates present on foods, such as for example fruits and vegetables, before ingestion, so as to avoid ingestion/accumulation by living beings.

This use can be exploited in a use in the reduction of the concentration of phosphonates in vitro, (not in vivo, on living beings). Such use may be provided for example when washing the food, before consumption thereof.

Furthermore, it was surprisingly observed that the isolated bacterial strains described herein, or mixtures thereof, or compositions thereof, may be used for the reduction of the amount of phosphonates previously ingested or accumulated - through food - in the animal or human microbiota.

It is clear that the isolated bacterial strains subject of the present invention, the mixtures thereof or the compositions thereof, may be used for the maintenance/restoration of the human microbiota homeostasis damaged by the accumulation of phosphonates.

Preferably, the present invention relates to the bacterial strain Lactobacillus brevis LBR01, identified under deposit number DSM 23034, for use in the maintenance/restoration of the human microbiota homeostasis damaged by the accumulation of phosphonates.

Such therapeutic and preventive use may be understood to be a prevention due to the intake of the isolated strain, or the mixture or the composition according to the invention before or during meals so as to avoid the accumulation of phosphonates in the organism. It may also be understood to be for restoration/relief purposes where a phosphonate accumulation is diagnosed due to daily ingestion of food containing phosphonates.

In this second case, regular intake of the isolated strain, or the mixture or the composition according to the invention, allows to reduce the concentration of phosphonates in the organism resulting in the restoration of the correct health condition of the microbiota and/or the organism.

In a preferred embodiment, said mixture comprises or alternatively consists of at least one strain belonging to the species Lactobacillus brevis, wherein said strain is selected from the group consisting of (i) Lactobacillus brevis LBR01, identified under deposit number DSM 23034, Lactobacillus brevis LBR02, identified under deposit number DSM 29404; and mixtures thereof.

Preferably, the mixture according to the present invention comprises or alternatively consists of an isolated bacterial strain belonging to the species Lactobacillus brevis LBR01, identified under deposit number DSM 23034.

In a preferred embodiment, the mixture comprises or, alternatively, consists of an isolated bacterial strain belonging to the species Lactobacillus brevis, preferably Lactobacillus brevis LBR01 (DSM 23034), preferably in a mixture with inulin or maltodextrin. This mixture is used for the uses and treatment methods as described in the present invention.

A preferred embodiment relates to the use - preferably non-therapeutic - of an isolated bacterial strain belonging to the species Lactobacillus brevis, said strain being the bacterial strain Lactobacillus brevis LBR01, identified under deposit number DSM 23034.

A preferred embodiment relates to the in vitro use for the reduction of the amount of phosphonates, of an isolated bacterial strain belonging to the species Lactobacillus brevis, said strain being the bacterial strain Lactobacillus brevis LBR01, identified under deposit number DSM 23034.

The use of such isolated strain, or said mixture (or said composition) is for the reduction of an ingested or accumulated amount of phosphonates in the human gut microbiota.

The phosphonates are preferably selected from the group comprising or, alternatively, consisting of methylphosphonate, ethylphosphonate, phosphonoacetate, phosphonopyruvate, 2-aminoethylphosphonate, glyphosate and mixtures thereof. More preferably, the phosphonates comprise or, alternatively, consist of 2-aminoethylphosphonate and/or glyphosate. By way of example, the glyphosate could be contained in a glyphosate-based herbicide (GBH).

Said at least one isolated bacterial strain belonging to the species Lactobacillus brevis is selected from the group comprising or, alternatively, consisting of the bacterial strains:
Lactobacillus brevis LBR01, identified under deposit number DSM 23034, deposited at the depository authority Deutsche Sammlung von Mikroorganismen und Zellkulturen GmbH (Germany) (DMSZ), on 13.10.2009, deposited by Probiotical S.p.A.;
Lactobacillus brevis LBR02, identified under deposit number DSM 29404, deposited at the depository authority Deutsche Sammlung von Mikroorganismen und Zellkulturen GmbH (Germany) (DMSZ), on 10.09.2014, deposited by Mofin Srl;
and mixtures thereof.

In an embodiment, said at least one isolated bacterial strain belonging to the species Lactobacillus plantarum and/or Lactobacillus pentosus is selected from the group comprising or, alternatively, consisting of the bacterial strains belonging to the species Lactobacillus plantarum and/or Lactobacillus pentosus and identified in Table 1 below, or mixtures thereof.

TABLE 1

| Strain name | Abbreviation | Depository authority | Deposit number | Date of deposit | Filing party |
|---|---|---|---|---|---|
| Lactobacillus plantarum 776/1 bi | LP 02 | BCCM LMG | LMG P-21020 | 16.10.2001 | Mofin Srl |
| Lactobacillus plantarum 476LL 20 bi | LP 01 | BCCM LMG | LMG P-21021 | 16.10.2001 | Mofin Srl |
| Lactobacillus plantarum PR ci | | BCCM LMG | LMG P-21022 | 16.10.2001 | Mofin Srl |
| Lactobacillus plantarum 776/2 hi | | BCCM LMG | LMG P-21023 | 16.10.2001 | Mofin Srl |
| Lactobacillus plantarum 501/2 gi | | BCCM LMG | LMG P-21385 | 31.01.2002 | Mofin Srl |
| Lactobacillus plantarum 501/4 li | | BCCM LMG | LMG P-21389 | 15.03.2002 | Mofin Srl |
| Lactobacillus plantarum | LP 07 | DSMZ | DSM 17104 | 01.02.2005 | Anidral Srl |
| Lactobacillus plantarum LP10 (1631) | | DSMZ | DSM 19071 | 21.02.2007 | Anidral Srl |
| Lactobacillus plantarum | LP1 | DSMZ | DSM 22107 | 10.12.2008 | Probiotical SpA |
| Lactobacillus plantarum | LP10 | DSMZ | DSM 29389 | 10.09.2014 | Mofin Srl |
| Lactobacillus plantarum | LP11 | DSMZ | DSM 29390 | 10.09.2014 | Mofin Srl |
| Lactobacillus plantarum | LP12 | DSMZ | DSM 29400 | 10.09.2014 | Mofin Srl |
| Lactobacillus plantarum | LP13 | DSMZ | DSM 29401 | 10.09.2014 | Mofin Srl |
| Lactobacillus plantarum | LP14 | DSMZ | DSM 33401 | 16.01.2020 | Probiotical SpA |
| Lactobacillus plantarum | LMC1 | DSMZ | DSM 32252 | 29.01.2016 | Probiotical SpA |
| Lactobacillus pentosus 9/1 ei | | BCCM LMG | LMG P-21019 | 16.10.2001 | Mofin Srl |
| Lactobacillus pentosus | LPS 01 | DSMZ | DSM 21980 | 14.11.2008 | Probiotical SpA |
| Lactobacillus pentosus | LPS 02 | DSMZ | DSM 26038 | 06.06.2012 | Probiotical SpA |
| Lactobacillus pentosus | LPS03 | DSMZ | DSM 29402 | 10.09.2014 | Mofin Srl |

In an embodiment, said at least one isolated bacterial strain belonging to the species Lactobacillus plantarum and/or Lactobacillus pentosus is selected from the group comprising or, alternatively, consisting of the bacterial strains belonging to the species Lactobacillus plantarum and/or Lactobacillus pentosus and identified in Table 2 below, or mixtures thereof.

TABLE 2

| Strain name | Abbreviation | Depository authority | Deposit number | Date of deposit | Filing party |
|---|---|---|---|---|---|
| Lactobacillus plantarum 776/1 bi | LP 02 | BCCM LMG | LMG P-21020 | 16.10.2001 | Mofin Srl |
| Lactobacillus plantarum 476LL 20 bi | LP 01 | BCCM LMG | LMG P-21021 | 16.10.2001 | Mofin Srl |
| Lactobacillus plantarum | LP14 | DSMZ | DSM 33401 | 16.01.2020 | Probiotical SpA |
| Lactobacillus pentosus | LPS 01 | DSMZ | DSM 21980 | 14.11.2008 | Probiotical SpA |

It should be observed that all the isolated bacterial strains reported above are deposited according to the Budapest Treaty. The proprietor of the present patent application grants consent to make the aforementioned bacterial strains available to the public, according to the provisions laid down by the treaty in question. More precisely, as pertains to the abbreviations reported in the depository authority column, the abbreviations below were used: BCCM LMG = Belgian Co-Ordinated Collections of Micro-Organisms, Laboratory of Microbiology (Belgium); DSMZ = Deutsche Sammlung von Mikroorganismen und Zellkulturen GmbH (Germany).

In an embodiment, said mixture (or said composition) comprises at least two, or at least three, isolated bacterial strains selected from said group of strains belonging to the species identified in Table 1 or Table 2.

The inventors of the present invention tested the bacteria of Table 1 and found that such bacteria have genes suitable to generate an enzyme kit for the degradation of phosphonates. For the sake of clarity of description and simplification, in the description below, specific reference will be made only to the bacteria of Table 2 with reference to FIG. 1, which exemplifies the position of the nucleotide sequences designated to encode the following proteins involved in the pathway of phosphonate metabolism:

Lactobacillus plantarum 476LL 20 bi (LP01; identified under deposit number LMG P-21021):
sequence 1: phosphonate ABC transporter substrate-binding protein (PhnD);
sequence 2: phosphonate ABC transporter ATP binding protein (PhnC);
sequence 3: phosphonate ABC transporter permease protein (PhnE2);
sequence 5: phosphonate ABC transporter permease protein (PhnE1);
sequence 7: phosphonoacetaldehyde hydrolase (EC 3.11.1.1);
sequence 9: 2 aminoethylphosphonate: pyruvate aminotrasferse (EC 2.6.1.37).

Lactobacillus plantarum 776/1 bi (LP02; identified under deposit number LMG P-21020):
sequence 1: phosphonate ABC transporter substrate-binding protein (PhnD);
sequence 2: phosphonate ABC transporter ATP binding protein (PhnC);
sequence 3: phosphonate ABC transporter permease protein (PhnE2);
sequence 5: phosphonate ABC transporter permease protein (PhnE1);
sequence 7: phosphonoacetaldehyde hydrolase (EC 3.11.1.1);
sequence 9: 2 aminoethylphosphonate: pyruvate aminotrasferse (EC 2.6.1.37).

Lactobacillus plantarum (LP14; identified under deposit number DSM 33401):
sequence 1: phosphonate ABC transporter substrate-binding protein (PhnD);
sequence 2: phosphonate ABC transporter ATP binding protein (PhnC);
sequence 3: phosphonate ABC transporter permease protein (PhnE2);
sequence 5: phosphonate ABC transporter permease protein (PhnE1);
sequence 7: phosphonoacetaldehyde hydrolase (EC 3.11.1.1);
sequence 9: 2 aminoethylphosphonate: pyruvate aminotrasferse (EC 2.6.1.37).

Lactobacillus pentosus (LPS01; identified under deposit number DSM 21980):
sequence 1: phosphonate ABC transporter substrate-binding protein (PhnD);
sequence 2: phosphonate ABC transporter ATP binding protein (PhnC);
sequence 3: phosphonate ABC transporter permease protein (PhnE2);
sequence 5: phosphonate ABC transporter permease protein (PhnE1);
sequence 8: phosphonoacetaldehyde hydrolase (EC 3.11.1.1);
sequence 10: 2 aminoethylphosphonate: pyruvate aminotrasferse (EC 2.6.1.37).

Taking the degradation of 2-aminoethylphosphonate for example, it has been observed that the aforementioned strains, whose sequences have been exemplified in FIG. 1, have genes capable of encoding the enzyme 2AEP-pyruvate aminotransferase (E.C. 2.6.1.37) which converts 2-aminoethylphosphonate into 2-phosphonoacetaldehyde. The enzyme phosphonoacetaldehyde-hydrolase (E.E. 3.11.1.1) - also encoded by the genes of the aforementioned isolated bacterial strains - converts 2-phosphonoacetaldehyde into acetaldehyde and phosphorus (P), thus completing the phosphatase pathway.

According to a particularly advantageous aspect, the strain Lactobacillus pentosus (LPS01; identified under deposit number DSM 21980) is characterised by the presence - on the genome - of portions encoding the protein phosphonate ABC transporter substrate-binding protein (PhnD, PhnC) and the protein phosphonate ABC transporter permease protein (PhnE2, PhnE1). The co-presence of the aforementioned proteins shows the ability of bacterial strains to be involved in the pathways of phosphonate metabolism.

In an embodiment, said mixture and said composition comprising said mixture, comprise two isolated bacterial strains belonging to the species Lactobacillus brevis, identified as Lactobacillus brevis LBR01 and Lactobacillus brevis LBR02, which are present in a CFU/g (or AFU/g) ratio comprised from 4:1 to 1:4, more preferably from 2:1 to 1:2, even more preferably substantially 1:1.

In an embodiment, said mixture and said composition comprising said mixture, comprise two isolated bacterial strains identified in Table 1, or in Table 2, which are present in a CFU/g (or AFU/g) ratio comprised from 4:1 to 1:4, more preferably from 2:1 to 1:2, even more preferably substantially 1:1.

In another embodiment, said mixture and said composition comprising said mixture, comprise three isolated bacterial strains identified in Table 1, or in Table 2, which are present in a CFU/g (or AFU/g) ratio comprised from 4:1:1 to 1:4:4, more preferably from 2:1:1 to 1:2:2, even more preferably substantially 1:1:1.

The isolated bacterial strain, or each isolated bacterial strain, is present in said mixture in a concentration comprised from 1×10^6 CFU/g (or AFU/g) to 1×10^12 CFU/g (or AFU/g) of mixture; preferably in a concentration comprised from 1×10^8 CFU/g (or AFU/g) to 1×10^10 CFU/g (or AFU/g) of mixture, for example in a concentration of about 1×10^9 CFU/g (or AFU/g) of mixture.

The “CFU” (colony-forming units) indicates the so-called units of colony-forming bacterial units. The “AFU” (active fluorescent units) are active fluorescent bacterial units - having an intact cell membrane and which therefore are viable cells - which can be identified through flow cytofluorometry as specified in the ISO 19344:2015(E) standard. Such standard is meant in the validity version at the priority date of the present patent application.

The isolated bacterial strain is preferably present as a culture of viable bacterial cells wherein the cells have an intact cell membrane; or said isolated bacterial strain is present as a culture of non-viable bacterial cells or cell components, or cell extracts, or cell lysates.

Once prepared, through a fermentation process carried out using the equipment and methods known to the man skilled in the art of fermentations, the at least one isolated bacterial strain may be used as such, in the solid form of flake, powder, granules or in the dried or freeze-dried form. Alternatively, said isolated strain may be mixed with an additive and/or technological excipient (cutting agent) such as for example inulin, maltodextrin or mixtures thereof.

Preferably, the technological additive (or the plurality thereof) may be present in a ratio - expressed as grams of additive on CFU/g (or AFU/g) of bacterial culture - [additive]:[bacterial culture] comprised between 0.1:30 and 1:1. More preferably, the technological additive (or the plurality thereof) may be present in an amount comprised from 0.01% to 50% by weight, with respect to the total weight of the mixture, preferably in an amount comprised from 0.05% to 30% by weight, with respect to the total weight of the mixture, more preferably in an amount comprised from 0.1% to 15% by weight, with respect to the total weight of the mixture, for example comprised from 0.15% to 10% by weight, or comprised from 0.2% to 5% by weight, or comprised from 0.3% to 2% by weight, with respect to the total weight of the mixture.

Maltodextrin (CAS No. 9050-36-6) is a complex, water-soluble carbohydrate obtained through chemical hydrolysis processes mainly from the breakdown of cereal starches (corn, oats, wheat, rice) or tuber starches (potatoes, tapioca). Preferably, in the mixture and in the composition, subject of the present invention, maltodextrin and/or inulin are in solid form, for example of powder or granulate.

Furthermore, forming an object of the present invention is a non-therapeutic use of a composition comprising said mixture, and at least one physiologically and/or pharmaceutically acceptable excipient or additive, in the reduction of an ingested or accumulated amount of phosphonates in the human microbiota. Preferably, the physiologically and/or pharmaceutically acceptable excipient or additive is selected from the group comprising or, alternatively, consisting of maltodextrin, inulin, and mixtures thereof; or, it may also be selected from the group consisting of a preservative and/or a stabilising substance, known and normally used in the industry, in addition to said maltodextrin, inulin, and mixtures thereof.

An [isolated bacterial strain]:[excipient] ratio - expressed as CFU/g (or AFU/g) of bacterial culture on grams of additive - is preferably comprised from 10:1 to 1:10, more preferably comprised from 5:1 to 1:5, even more preferably comprised from 2:1 to 1:2.

Furthermore, forming an object of the present invention is a mixture and a composition comprising said mixture, said mixture comprising or, alternatively, consisting of at least one isolated bacterial strain belonging to the species Lactobacillus brevis, at least one isolated bacterial strain belonging to the species Lactobacillus plantarum and/or at least one isolated bacterial strain belonging to the species Lactobacillus pentosus for use in a method for the preventive or curative treatment of a disorder or an ailment or a disease associated with an ingestion or with an accumulation - through food - of phosphonates in the human or animal microbiota.

Said disorder or said ailment or said disease is selected from the group comprising or, alternatively, consisting of: coeliac disease, heart diseases, depression, autism, cancer, non-alcoholic fatty liver disease or neural disease, diabetes, obesity, asthma, chronic obstructive pulmonary disease (COPD), pulmonary oedema, adrenal insufficiency, hypothyroidism, Alzheimer’s disease, amyotrophic lateral sclerosis (ALS), Parkinson’s disease, prion diseases, lupus, mitochondrial disease, non-Hodgkin’s lymphoma, spinal defects, spina bifida, sterility, hypertension, glaucoma, osteoporosis and renal insufficiency.

It has been observed that phosphonates in the organism could result in several diseases as reported above. As a result, the isolated bacterial strains described herein, the mixtures thereof I or the compositions comprising them may be used for the treatment or prevention of diseases caused by an accumulation of phosphonates in the microbiota such as, Parkinson’s syndrome, Alzheimer’s disease and Amyotrophic Lateral Sclerosis (ALS).

Furthermore, forming an object of the present invention is a mixture and a composition comprising said mixture, said mixture comprising or, alternatively, consisting of at least one isolated bacterial strain belonging to the species Lactobacillus brevis, (preferably Lactobacillus brevis LBR01, identified under deposit number DSMZ 23034), at least one isolated bacterial strain belonging to the species Lactobacillus plantarum and/or at least one isolated bacterial strain belonging to the species Lactobacillus pentosus for use in the maintenance/restoration of the homeostasis lost due to an accumulation of phosphonates ingested through food.

### EXPERIMENTAL PART

The present study shows the main results obtained regarding the ability of the strains subject of the present invention to reduce the amount of phosphonates, specifically of glyphosate.

Listed in table 3 are the strains used in the experiment, the growth medium and the concentration of glyphosate.

TABLE 3

List of strains, growth medium and concentrations of glyphosate tested

| | | | | |
|---|---|---|---|---|
| Micro-organisms | Growth medium | [Glyphosate] (mg/L) | | |
| L. plantarum LP01 | MRS | 0.3 | 1.2 | 2.5 |
| L. plantarum LP02 | MRS | | | |
| L. plantarum LP14 | MRS | | | |
| L. pentosus LPS01 | MRS | | | |
| L. brevis LBR01 | TIL | | | |

The reduction action of the amount of phosphonates was evaluated by exposing the bacterial strains according to the present invention to glyphosate for 24 hours following the two methods schematised in FIG. 2.

Method A: simultaneous growth of bacterial strains subject of the present invention in liquid medium and exposure to glyphosate for 24 hours, in order to evaluate the reduction action of phosphonates (glyphosate) which occurs during the growth of the bacterial strains according to the present invention (FIG. 2A).

Method B: growth - for 24 hours - of the bacterial strains according to the present invention in a glyphosate-free liquid medium and subsequent exposure of the latter (by adding them to the medium) for 24 hours, in order to evaluate the reduction of phosphonates (glyphosate) in the medium after accumulating the biomass of bacterial strains according to the present invention (FIG. 2B).

### Preparation of Working Cell Banks

Working cell banks were prepared from a homogeneous cell suspension obtained by inoculating 100 µL of the glycerinates of the strains reported in Table 3 in 15 mL-tubes containing 10 mL of medium and incubating overnight at 37° C. without stirring.

Specifically, the glycerinates were prepared as follows:
67.5% vol cell culture;
12.5% vol glycerol;
20% vol sucrose, at the initial concentration of 100 mg/mL.

### GROWTH KINETICS

Starting from the generated working cell banks, as described above, 500 µL of bacterial strains according to the present invention were pre-inoculated in 50 mL-tubes, containing 50 mL of growth medium.

Following incubation overnight at 37° C. without stirring, the strains were inoculated to a starting OD600 equal to 0.2 in 15 mL-tubes containing 10 mL of medium.

Pre-inocula and inocula were carried out so as to minimise the air volume in the samples.

Glyphosate was added following the two methods (A and B) described in FIG. 2.

### QUANTIFICATION OF GLYPHOSATE

Glyphosate was quantified using the ELISA kit by means of an immunosorbent assay. In particular, with regard to “Method A” (FIG. 2A) samples were analysed at times T0 and T24* while with regard to “Method B” (FIG. 2B) those at times T24, after adding glyphosate, and T24*.

Besides being particularly sensitive, actually allowing to quantify glyphosate at concentrations in the order of ppbs, this method is also recommended, due to its high selectivity, for the analysis of complex matrices such as soil and food samples.

### RESULTS

### Evaluation of the Reduction Action of the Amount of Phosphonates (Glyphosate) by the Bacterial Strains Described in the Present Invention

As described previously, the reduction activity of the amount of glyphosate of the probiotic strains LP01, LP02, LP14, LPS01, LBR01 was evaluated using the ELISA immunosorbent assay evaluating 3 different concentrations: 2.5 - 1.2 - 0.3 mg/L.

Table 4 reports the results obtained following the two methods of administration, A and B as reported in FIG. 2 and in the corresponding description.

The inhibition percentages were calculated for each condition by comparing the concentration of glyphosate at the end of the test with the one at the respective time 0.

TABLE 4

results of the quantification of glyphosate using the ELISA immunosorbent assay in the two types of exposure, Experiment A (EXP A), Experiment B (EXP B). The decrease % of glyphosate are also reported
LBR01

| EXP A | | | EXP B | | |
|---|---|---|---|---|---|
| [Glyphosate] | mg/L | % | [Glyphosate] | mg/L | % |
| T0 | T24* | Decrease | T24 | T24* | Decrease |
| 2.33 | 2.10 | 10% | 2.41 | 2.47 | - |
| 1.19 | 1.08 | 9% | 1.25 | 1.11 | 11% |
| 0.33 | 0.30 | 10% | 0.31 | 0.27 | 12% |

As highlighted in Table 4, the probiotic strain LBR01 is capable of determining a reduction of glyphosate within the 24 hours of exposure. Specifically, the greater percentage decrease, equal to 12%, is observed when glyphosate is administered at the concentration of 0.3 mg/L following “Method B”, that is by adding glyphosate to the growth medium only after the initial 24-hour growth phase without the contaminant.

This condition was then also tested by conducting the triplicate test and extending the step of exposure to glyphosate to 72 hours (Table 5).

The data obtained confirm that the strain LBR01 is capable of reducing the concentration of glyphosate in the growth medium.

TABLE 5

Results of the quantification of glyphosate using ELISA immunosorbent assay in the type of experiment B (EXP B), wherein exposure was extended to 72 hours, for the strain LBR01. Furthermore, the standard deviations and the % decrease of glyphosate are reported
LBR01
EXP B

| [Glysophate] mg/L | | % decrease |
|---|---|---|
| T24 | T72* | |
| 0.346±0.007 | 0.326±0.008 | 6% |

Furthermore, the following embodiments are part of the present invention:

FR1. A mixture comprising or, alternatively, consisting of at least one isolated bacterial strain belonging to the species Lactobacillus plantarum and/or at least one isolated bacterial strain belonging to the species Lactobacillus pentosus for use in a method for the preventive or curative treatment of a disorder or an ailment or a disease associated with an ingestion or an accumulation of phosphonates in human microbiota.

FR2. The mixture for use according to FR1, wherein the disorder or ailment or said disease is selected from the group comprising or, alternatively, consisting of: non-alcoholic fatty liver disease or neural disease, diabetes, obesity, asthma, chronic obstructive pulmonary disease (COPD), pulmonary oedema, adrenal insufficiency, hypothyroidism, Alzheimer’s disease, amyotrophic lateral sclerosis (ALS), Parkinson’s disease, prion diseases, lupus, mitochondrial disease, non-Hodgkin’s lymphoma, spinal defects, spina bifida, sterility, hypertension, glaucoma, osteoporosis and renal insufficiency.

FR3. The mixture for use according to any one of FR1-2, wherein said at least one isolated bacterial strain is selected from the group comprising or, alternatively, consisting of:

| Strain name | Abbreviation | Depository authority | Deposit number | Date of deposit |
|---|---|---|---|---|
| Lactobacillus plantarum 776/1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum PR ci | | BCCM LMG | LMG P-21022 | 16.10.2001 |
| Lactobacillus plantarum 776/2 hi | | BCCM LMG | LMG P-21023 | 16.10.2001 |
| Lactobacillus plantarum 501/2 gi | | BCCM LMG | LMG P-21385 | 31.01.2002 |
| Lactobacillus plantarum 501/4 li | | BCCM LMG | LMG P-21389 | 15.03.2002 |
| Lactobacillus plantarum | LP 07 | DSMZ | DSM 17104 | 01.02.2005 |
| Lactobacillus plantarum LP 10 (1631) | | DSMZ | DSM 19071 | 21.02.2007 |
| Lactobacillus plantarum | LP1 | DSMZ | DSM 22107 | 10.12.2008 |
| Lactobacillus plantarum | LP 09 | DSMZ | DSM 25710 | 24.02.2012 |
| Lactobacillus plantarum | LP10 | DSMZ | DSM 29389 | 10.09.2014 |
| Lactobacillus plantarum | LP11 | DSMZ | DSM 29390 | 10.09.2014 |
| Lactobacillus plantarum | LP12 | DSMZ | DSM 29400 | 10.09.2014 |
| Lactobacillus plantarum | LP13 | DSMZ | DSM 29401 | 10.09.2014 |
| Lactobacillus plantarum | LP14 | DSMZ | DSM 33401 | 16.01.2020 |
| Lactobacillus plantarum | LMC1 | DSMZ | DSM 32252 | 29.01.2016 |
| Lactobacillus pentosus 9/1 ei | | BCCM LMG | LMG P-21019 | 16.10.2001 |
| Lactobacillus pentosus | LPS 01 | DSMZ | DSM 21980 | 14.11.2008 |
| Lactobacillus pentosus | LPS 02 | DSMZ | DSM 26038 | 06.06.2012 |
| Lactobacillus pentosus | LPS03 | DSMZ | DSM 29402 | 10.09.2014 |

and combinations thereof.

FR4. The mixture for use according to FR3, wherein said at least one isolated bacterial strain is selected from the group comprising or, alternatively, consisting of:

| Strain name | Abbreviation | Depository authority | Deposit number | Date of deposit |
|---|---|---|---|---|
| Lactobacillus plantarum 776/1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum | LP14 | DSMZ | DSM 33401 | 16.01.2020 |
| Lactobacillus pentosus | LPS 01 | DSMZ | DSM 21980 | 14.11.2008 |

and combinations thereof.

FR5. The mixture for use according to any one of FR1-4, wherein said mixture comprises or, alternatively, consists of at least two or at least three isolated bacterial strains.

FR6. The mixture for use according to any one of FR1-5, wherein said phosphonates are selected from the group comprising or, alternatively, consisting of methylphosphonate, ethylphosphonate, phosphonoacetate, phosphonopyruvate, 2-aminoethylphosphonate, glyphosate and mixtures thereof, preferably 2-aminoethylphosphonate or glyphosate.

FR7. The mixture for use according to any one of FR1-6, wherein the glyphosate is contained in a glyphosate-based herbicide (GBH).

FR8. Non-therapeutic use of a mixture comprising or, alternatively, consisting of at least one isolated bacterial strain belonging to the species Lactobacillus plantarum and/or at least one isolated bacterial strain belonging to the species Lactobacillus pentosus in the reduction of an ingested or accumulated amount of phosphonates in the human microbiota.

FR9. The non-therapeutic use of the mixture according to FR8, wherein said at least one isolated bacterial strain is selected from the group comprising or, alternatively, consisting of:

| Strain name | Abbreviation | Depository authority | Deposit number | Date of deposit |
|---|---|---|---|---|
| Lactobacillus plantarum 776/1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum PR ci | | BCCM LMG | LMG P-21022 | 16.10.2001 |
| Lactobacillus plantarum 776/2 hi | | BCCM LMG | LMG P-21023 | 16.10.2001 |
| Lactobacillus plantarum 501/2 gi | | BCCM LMG | LMG P-21385 | 31.01.2002 |
| Lactobacillus plantarum 501/4 li | | BCCM LMG | LMG P-21389 | 15.03.2002 |
| Lactobacillus plantarum | LP 07 | DSMZ | DSM 17104 | 01.02.2005 |
| Lactobacillus plantarum LP 10 (1631) | | DSMZ | DSM 19071 | 21.02.2007 |
| Lactobacillus plantarum | LP1 | DSMZ | DSM 22107 | 10.12.2008 |
| Lactobacillus plantarum | LP10 | DSMZ | DSM 29389 | 10.09.2014 |
| Lactobacillus plantarum | LP11 | DSMZ | DSM 29390 | 10.09.2014 |
| Lactobacillus plantarum | LP12 | DSMZ | DSM 29400 | 10.09.2014 |
| Lactobacillus plantarum | LP13 | DSMZ | DSM 29401 | 10.09.2014 |
| Lactobacillus plantarum | LP14 | DSMZ | DSM 33401 | 16.01.2020 |
| Lactobacillus plantarum | LMC1 | DSMZ | DSM 32252 | 29.01.2016 |
| Lactobacillus pentosus 9/1 ei | | BCCM LMG | LMG P-21019 | 16.10.2001 |
| Lactobacillus pentosus | LPS 01 | DSMZ | DSM 21980 | 14.11.2008 |
| Lactobacillus pentosus | LPS 02 | DSMZ | DSM 26038 | 06.06.2012 |
| Lactobacillus pentosus | LPS03 | DSMZ | DSM 29402 | 10.09.2014 |

and combinations thereof; or, alternatively, wherein said at least one isolated bacterial strain is selected from the group comprising or, alternatively, consisting of:

| Name | Abbreviation | Depository authority | Deposit number | Date of deposit |
|---|---|---|---|---|
| Lactobacillus plantarum 776/1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum | LP14 | DSMZ | DSM 33401 | 16.01.2020 |
| Lactobacillus pentosus | LPS 01 | DSMZ | DSM 21980 | 14.11.2008 |

and combinations thereof.

FR10. The non-therapeutic use according to FR8 or9, wherein the phosphonates are selected from the group comprising or, alternatively, consisting of methylphosphonate, ethylphosphonate, phosphonoacetate, phosphonopyruvate, 2-aminoethylphosphonate, glyphosate and mixtures thereof, preferably 2-aminoethylphosphonate or glyphosate; preferably wherein the glyphosate is contained in glyphosate-based herbicide (GBH).

Further embodiments of the present invention (FR-Bn) are reported below:

FR-B1. A mixture comprising or, alternatively, consisting of at least one isolated bacterial strain belonging to the species:
(i) Lactobacillus brevis, wherein said strain is selected from the group consisting or, alternatively, comprising of Lactobacillus brevis LBR01, identified under deposit number DSM 23034, Lactobacillus brevis LBR02, identified under deposit number DSM 29404; and mixtures thereof;
optionally combined with at least one isolated bacterial strain belonging to the species Lactobacillus plantarum and/or at least one isolated bacterial strain belonging to the species Lactobacillus pentosus for use in a method for the preventive or curative treatment of a disorder or an ailment or a disease associated with an ingestion or an accumulation of phosphonates in the human microbiota.

FR-B2. The mixture for use according to FR-B 1, wherein said at least one isolated bacterial strain belonging to the species (i) Lactobacillus brevis, is the bacterial strain Lactobacillus brevis LBR01, identified under deposit number DSM 23034.

FR-B3. The mixture for use according to any one of the preceding FR-Bn, wherein said disorder or ailment or said disease is selected from the group comprising or, alternatively, consisting of: non-alcoholic fatty liver disease or neural disease, diabetes, obesity, asthma, chronic obstructive pulmonary disease (COPD), pulmonary oedema, adrenal insufficiency, hypothyroidism, Alzheimer’s disease, amyotrophic lateral sclerosis (ALS), Parkinson’s disease, prion diseases, lupus, mitochondrial disease, non-Hodgkin’s lymphoma, spinal defects, spina bifida, sterility, hypertension, glaucoma, osteoporosis and renal insufficiency.

FR-B4. The mixture for use according to any one of the preceding FR-Bn, wherein said at least one isolated bacterial strain belonging to the species Lactobacillus plantarum and/or at least one isolated bacterial strain belonging to the species Lactobacillus pentosus is selected from the group comprising or, alternatively, consisting of:

| Strain name | Abbreviation | Depository authority | Deposit number | Date of deposit |
|---|---|---|---|---|
| Lactobacillus plantarum 776/1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum PR ci | | BCCM LMG | LMG P-21022 | 16.10.2001 |
| Lactobacillus plantarum 776/2 hi | | BCCM LMG | LMG P-21023 | 16.10.2001 |
| Lactobacillus plantarum 501/2 gi | | BCCM LMG | LMG P-21385 | 31.01.2002 |
| Lactobacillus plantarum 501/4 li | | BCCM LMG | LMG P-21389 | 15.03.2002 |
| Lactobacillus plantarum | LP 07 | DSMZ | DSM 17104 | 01.02.2005 |
| Lactobacillus plantarum LP 10 (1631) | | DSMZ | DSM 19071 | 21.02.2007 |
| Lactobacillus plantarum | LP1 | DSMZ | DSM 22107 | 10.12.2008 |
| Lactobacillus plantarum | LP 09 | DSMZ | DSM 25710 | 24.02.2012 |
| Lactobacillus plantarum | LP10 | DSMZ | DSM 29389 | 10.09.2014 |
| Lactobacillus plantarum | LP11 | DSMZ | DSM 29390 | 10.09.2014 |
| Lactobacillus plantarum | LP12 | DSMZ | DSM 29400 | 10.09.2014 |
| Lactobacillus plantarum | LP13 | DSMZ | DSM 29401 | 10.09.2014 |
| Lactobacillus plantarum | LP14 | DSMZ | DSM 33401 | 16.01.2020 |
| Lactobacillus plantarum | LMC1 | DSMZ | DSM 32252 | 29.01.2016 |
| Lactobacillus pentosus 9/1 ei | | BCCM LMG | LMG P-21019 | 16.10.2001 |
| Lactobacillus pentosus | LPS 01 | DSMZ | DSM 21980 | 14.11.2008 |
| Lactobacillus pentosus | LPS 02 | DSMZ | DSM 26038 | 06.06.2012 |
| Lactobacillus pentosus | LPS03 | DSMZ | DSM 29402 | 10.09.2014 |

and combinations thereof.

FR-B5. The mixture for use according to FR-B4 wherein said at least one isolated bacterial strain belonging to the species Lactobacillus plantarum and/or at least one isolated bacterial strain belonging to the species Lactobacillus pentosus is selected from the group comprising or, alternatively, consisting of:

| Strain name | Abbreviation | Depository authority | Deposit number | Date of deposit |
|---|---|---|---|---|
| Lactobacillus plantarum 776/1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum | LP14 | DSMZ | DSM 33401 | 16.01.2020 |
| Lactobacillus pentosus | LPS 01 | DSMZ | DSM 21980 | 14.11.2008 |

and combinations thereof.

FR-B6. The mixture for use according to any one of FR-B1-5, wherein said phosphonates are selected from the group comprising or, alternatively, consisting of methylphosphonate, ethylphosphonate, phosphonoacetate, phosphonopyruvate, 2-aminoethylphosphonate, glyphosate and mixtures thereof, preferably 2-aminoethylphosphonate or glyphosate.

FR-B7. The mixture for use according to any one of FR-B1-6, wherein the glyphosate is contained in a glyphosate-based herbicide (GBH).

FR-B8. A non-therapeutic use - in the reduction of an ingested or accumulated amount of phosphonates in the human microbiota - of a mixture comprising or, alternatively, consisting of at least one isolated bacterial strain belonging to the species:
(i) Lactobacillus brevis, wherein said strain is selected from the group consisting or, alternatively, comprising of Lactobacillus brevis LBR01, identified under deposit number DSM 23034, Lactobacillus brevis LBR02, identified under deposit number DSM 29404; and mixtures thereof;
optionally combined with at least one isolated bacterial strain belonging to the species Lactobacillus plantarum and/or at least one isolated bacterial strain belonging to the species Lactobacillus pentosus.

FR-B9. The non-therapeutic use of the mixture according to FR-B8, wherein said at least one isolated bacterial strain belonging to the species Lactobacillus plantarum and/or at least one isolated bacterial strain belonging to the species Lactobacillus pentosus.is selected from the group comprising or, alternatively, consisting of:

| Strain name | Abbreviation | Depository authority | Deposit number | Date of deposit |
|---|---|---|---|---|
| Lactobacillus plantarum 776/1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum PR ci | | BCCM LMG | LMG P-21022 | 16.10.2001 |
| Lactobacillus plantarum 776/2 hi | | BCCM LMG | LMG P-21023 | 16.10.2001 |
| Lactobacillus plantarum 501/2 gi | | BCCM LMG | LMG P-21385 | 31.01.2002 |
| Lactobacillus plantarum 501/4 li | | BCCM LMG | LMG P-21389 | 15.03.2002 |
| Lactobacillus plantarum | LP 07 | DSMZ | DSM 17104 | 01.02.2005 |
| Lactobacillus plantarum LP 10 (1631) | | DSMZ | DSM 19071 | 21.02.2007 |
| Lactobacillus plantarum | LP1 | DSMZ | DSM 22107 | 10.12.2008 |
| Lactobacillus plantarum | LP10 | DSMZ | DSM 29389 | 10.09.2014 |
| Lactobacillus plantarum | LP11 | DSMZ | DSM 29390 | 10.09.2014 |
| Lactobacillus plantarum | LP12 | DSMZ | DSM 29400 | 10.09.2014 |
| Lactobacillus plantarum | LP13 | DSMZ | DSM 29401 | 10.09.2014 |
| Lactobacillus plantarum | LP14 | DSMZ | DSM 33401 | 16.01.2020 |
| Lactobacillus plantarum | LMC1 | DSMZ | DSM 32252 | 29.01.2016 |
| Lactobacillus pentosus 9/1 ei | | BCCM LMG | LMG P-21019 | 16.10.2001 |
| Lactobacillus pentosus | LPS 01 | DSMZ | DSM 21980 | 14.11.2008 |
| Lactobacillus pentosus | LPS 02 | DSMZ | DSM 26038 | 06.06.2012 |
| Lactobacillus pentosus | LPS03 | DSMZ | DSM 29402 | 10.09.2014 |

and combinations thereof; or, alternatively, wherein said at least one isolated bacterial strain is selected from the group comprising or, alternatively, consisting of:

| Name | Abbreviation | Depository authority | Deposit number | Date of deposit |
|---|---|---|---|---|
| Lactobacillus plantarum 776/1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum | LP14 | DSMZ | DSM 33401 | 16.01.2020 |
| Lactobacillus pentosus | LPS 01 | DSMZ | DSM 21980 | 14.11.2008 |

and combinations thereof.

FR-B10. The non-therapeutic use according to FR-B8 or9, wherein the phosphonates are selected from the group comprising or, alternatively, consisting of methylphosphonate, ethylphosphonate, phosphonoacetate, phosphonopyruvate, 2-aminoethylphosphonate, glyphosate and mixtures thereof, preferably 2-aminoethylphosphonate or glyphosate; preferably wherein the glyphosate is contained in glyphosate-based herbicide (GBH).

FR-B11. In vitro use - in the reduction of phosphonates, preferably glyphosate - of a mixture comprising or, alternatively, consisting of at least one isolated bacterial strain belonging to the species

(i) Lactobacillus brevis, wherein said strain is selected from the group consisting or, alternatively, comprising of Lactobacillus brevis LBR01, identified under deposit number DSM 23034, Lactobacillus brevis LBR02, identified under deposit number DSM 29404; and mixtures thereof.

FR-B12. The use according to FR-B11 where said reduction of phosphonates is comprised from 2% to 20%, preferably comprised from 2% to 14% with respect to the initial amount of phosphonates present.

FR-B13. A mixture comprising or, alternatively, consisting of at least one isolated bacterial strain belonging to the species:

(i) Lactobacillus brevis, wherein said strain is selected from the group comprising or, alternatively, consisting of Lactobacillus brevis LBR01, identified under deposit number DSM 23034, Lactobacillus brevis LBR02, identified under deposit number DSM 29404; and mixtures thereof, for use in the maintenance/restoration of the human microbiota homeostasis.

FR-B14. The mixture for use according to FR-B13, wherein said use is in the treatment of disorders related with loss of the human microbiota homeostasis, due to the accumulation/presence of phosphonates, such as for example glyphosate.

FR-B15. The mixture for use according to any one of FR-B13-14, wherein said phosphonates are selected from the group comprising or, alternatively, consisting of methylphosphonate, ethylphosphonate, phosphonoacetate, phosphonopyruvate, 2-aminoethylphosphonate, glyphosate and mixtures thereof, preferably 2-aminoethylphosphonate or glyphosate.

FR-B16. The mixture for use according to FR-B15, wherein the glyphosate is contained a glyphosate-based herbicide (GBH).

1. A method for treating a disorder or an ailment or a disease associated with ingestion or accumulation of phosphonates in the human microbiota, comprising:
administering to a subject a mixture comprising at least one isolated bacterial strain belonging to the species
Lactobacillus brevis, wherein said strain is selected from the group consisting of Lactobacillus brevis LBR01, identified under deposit number DSM 23034, Lactobacillus brevis LBR02, identified under deposit number DSM 29404; and mixtures thereof;
optionally combined with at least one isolated bacterial strain belonging to the species Lactobacillus plantarum and/or at least one isolated bacterial strain belonging to the species Lactobacillus pentosus.

2. The method according to claim 1, wherein said at least one isolated bacterial strain belonging to the species Lactobacillus brevis, is the bacterial strain Lactobacillus brevis LBR01, identified under deposit number DSM 23034.

3. The method according to claim 1, wherein said disorder or ailment or said disease is selected from the group comprising or, alternatively, consisting of: non-alcoholic fatty liver disease or neural disease, diabetes, obesity, asthma, chronic obstructive pulmonary disease (COPD), pulmonary oedema, adrenal insufficiency, hypothyroidism, Alzheimer’s disease, amyotrophic lateral sclerosis (ALS), Parkinson’s disease, prion diseases, lupus, mitochondrial disease, non-Hodgkin’s lymphoma, spinal defects, spina bifida, sterility, hypertension, glaucoma, osteoporosis, and renal insufficiency.

4. The method according to claim 1, wherein said at least one isolated bacterial strain belonging to the species Lactobacillus plantarum and/or at least one isolated bacterial strain belonging to the species Lactobacillus pentosus is selected from the following table:

| Strain name | Abbreviation | Depository authority | Deposit number | Date of deposit |
|---|---|---|---|---|
| Lactobacillus plantarum 776 /1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum PR ci | | BCCM LMG | LMG P-21022 | 16.10.2001 |
| Lactobacillus plantarum 776 /2 hi | | BCCM LMG | LMG P-21023 | 16.10.2001 |
| Lactobacillus plantarum 501/2 gi | | BCCM LMG | LMG P-21385 | 31.01.2002 |
| Lactobacillus plantarum 501/4 li | | BCCM LMG | LMG P-21389 | 15.03.2002 |
| Lactobacillus plantarum | LP 07 | DSMZ | DSM 17104 | 01.02.2005 |
| Lactobacillus plantarum LP 10 (1631) | | DSMZ | DSM 19071 | 21.02.2007 |
| Lactobacillus plantarum | LP1 | DSMZ | DSM 22107 | 10.12.2008 |
| Lactobacillus plantarum | LP 09 | DSMZ | DSM 25710 | 24.02.2012 |
| Lactobacillus plantarum | LP10 | DSMZ | DSM 29389 | 10.09.2014 |
| Lactobacillus plantarum | LP11 | DSMZ | DSM 29390 | 10.09.2014 |
| Lactobacillus plantarum | LP12 | DSMZ | DSM 29400 | 10.09.2014 |
| Lactobacillus plantarum | LP13 | DSMZ | DSM 29401 | 10.09.2014 |
| Lactobacillus plantarum | LP14 | DSMZ | DSM 33401 | 16.01.2020 |
| Lactobacillus plantarum | LMC1 | DSMZ | DSM 32252 | 29.01.2016 |
| Lactobacillus pentosus 9 /1 ei | | BCCM LMG | LMG P-21019 | 16.10.2001 |
| Lactobacillus pentosus | LPS 01 | DSMZ | DSM 21980 | 14.11.2008 |
| Lactobacillus pentosus | LPS 02 | DSMZ | DSM 26038 | 06.06.2012 |
| Lactobacillus pentosus | LPS03 | DSMZ | DSM 29402 | 10.09.2014 |

and combinations thereof.

5. The method according to claim 4, wherein said at least one isolated bacterial strain belonging to the species Lactobacillus plantarum and/or at least one isolated bacterial strain belonging to the species Lactobacillus pentosus is selected from the following table:

| Strain name | Abbreviation | Depository authority | Deposit number | Date of deposit |
|---|---|---|---|---|
| Lactobacillus plantarum 776 /1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum | LP14 | DSMZ | DSM 33401 | 16.01.2020 |
| Lactobacillus pentosus | LPS 01 | DSMZ | DSM 21980 | 14.11.2008 |

and combinations thereof.

6. The method according to claim 1, wherein said phosphonates are selected from the group comprising or, alternatively, consisting of methylphosphonate, ethylphosphonate, phosphonoacetate, phosphonopyruvate, 2-aminoethylphosphonate, glyphosate and mixtures thereof, preferably 2-aminoethylphosphonate or glyphosate.

7. The method according to claim 1, wherein the glyphosate is contained in a glyphosate-based herbicide (GBH).

8. A non-therapeutic use - in the reduction of an ingested or accumulated amount of phosphonates in the human microbiota - of a mixture comprising or, alternatively, consisting of at least one isolated bacterial strain belonging to the species:
(i) Lactobacillus brevis, wherein said strain is selected from the group consisting or, alternatively, comprising of Lactobacillus brevis LBR01, identified under deposit number DSM 23034, Lactobacillus brevis LBR02, identified under deposit number DSM 29404; and mixtures thereof;
optionally combined with at least one isolated bacterial strain belonging to the species Lactobacillus plantarum and/or at least one isolated bacterial strain belonging to the species Lactobacillus pentosus.

9. The non-therapeutic use of the mixture according to claim 8, wherein said at least one isolated bacterial strain belonging to the species Lactobacillus plantarum and/or at least one isolated bacterial strain belonging to the species Lactobacillus pentosus. is selected from the group comprising or, alternatively, consisting of:

| Strain name | Abbreviation | Depository authority | Deposit number | Date of deposit |
|---|---|---|---|---|
| Lactobacillus plantarum 776 /1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum PR ci | | BCCM LMG | LMG P-21022 | 16.10.2001 |
| Lactobacillus plantarum 776 /2 hi | | BCCM LMG | LMG P-21023 | 16.10.2001 |
| Lactobacillus plantarum 501/2 gi | | BCCM LMG | LMG P-21385 | 31.01.2002 |
| Lactobacillus plantarum 501/4 li | | BCCM LMG | LMG P-21389 | 15.03.2002 |
| Lactobacillus plantarum | LP 07 | DSMZ | DSM 17104 | 01.02.2005 |
| Lactobacillus plantarum LP 10 (1631) | | DSMZ | DSM 19071 | 21.02.2007 |
| Lactobacillus plantarum | LP1 | DSMZ | DSM 22107 | 10.12.2008 |
| Lactobacillus plantarum | LP10 | DSMZ | DSM 29389 | 10.09.2014 |
| Lactobacillus plantarum | LP11 | DSMZ | DSM 29390 | 10.09.2014 |
| Lactobacillus plantarum | LP12 | DSMZ | DSM 29400 | 10.09.2014 |
| Lactobacillus plantarum | LP13 | DSMZ | DSM 29401 | 10.09.2014 |
| Lactobacillus plantarum | LP14 | DSMZ | DSM 33401 | 16.01.2020 |
| Lactobacillus plantarum | LMC1 | DSMZ | DSM 32252 | 29.01.2016 |
| Lactobacillus pentosus 9/1 ei | | BCCM LMG | LMG P-21019 | 16.10.2001 |
| Lactobacillus pentosus | LPS 01 | DSMZ | DSM 21980 | 14.11.2008 |
| Lactobacillus pentosus | LPS 02 | DSMZ | DSM 26038 | 06.06.2012 |
| Lactobacillus pentosus | LPS03 | DSMZ | DSM 29402 | 10.09.2014 |

and combinations thereof; or, alternatively, wherein said at least one isolated bacterial strain is selected from the group comprising or, alternatively, consisting of:

| Name | Abbreviation | Depository authority | Deposit number | Date of deposit |
|---|---|---|---|---|
| Lactobacillus plantarum 776 /1 bi | LP 02 | BCCM LMG | LMG P-21020 | 16.10.2001 |
| Lactobacillus plantarum 476LL 20 bi | LP 01 | BCCM LMG | LMG P-21021 | 16.10.2001 |
| Lactobacillus plantarum | LP14 | DSMZ | DSM 33401 | 16.01.2020 |
| Lactobacillus pentosus | LPS 01 | DSMZ | DSM 21980 | 14.11.2008 |

and combinations thereof.

10. The non-therapeutic use according to claims 8, wherein the phosphonates are selected from the group comprising or, alternatively, consisting of methylphosphonate, ethylphosphonate, phosphonoacetate, phosphonopyruvate, 2-aminoethylphosphonate, glyphosate and mixtures thereof, preferably 2-aminoethylphosphonate or glyphosate; preferably wherein glyphosate is contained in a glyphosate-based herbicide (GBH).

11. In vitro use - in the reduction of phosphonates, preferably glyphosate - of a mixture comprising or, alternatively, consisting of at least one isolated bacterial strain belonging to the species (i) Lactobacillus brevis, wherein said strain is selected from the group consisting or, alternatively, comprising of Lactobacillus brevis LBR01, identified under deposit number DSM 23034, Lactobacillus brevis LBR02, identified under deposit number DSM 29404; and mixtures thereof.

12-13. (canceled)

14. The mixture for use according to claim 13, wherein said use is in the treatment of disorders related with loss of human microbiota homeostasis due to the accumulation/presence of phosphonates, such as for example glyphosate.

15. The mixture for use according to claim 13, wherein said phosphonates are selected from the group comprising or, alternatively, consisting of methylphosphonate, ethylphosphonate, phosphonoacetate, phosphonopyruvate, 2-aminoethylphosphonate, glyphosate and mixtures thereof, preferably 2-aminoethylphosphonate or glyphosate.

16. The mixture for use according to claim 15, wherein glyphosate is contained in a glyphosate-based herbicide (GBH).
